(19) United States
(12) Plant Patent Application Publication
(10) Pub. No.: US 2014/0325724 P1
(43) Pub. Date: Oct. 30, 2014

(54) Lavandula plant named 'Voodoo'

(50) ;

(71) Applicants: Luen Miller, Santa Cruz, CA (US); Manuel Morales, Royaloaks, CA (US)

(72) Inventors: Luen Miller, Santa Cruz, CA (US); Manuel Morales, Royaloaks, CA (US)

(21) Appl. No.: 13/986,394

(22) Filed: Apr. 26, 2013

(51) Int. Cl. A01H 5/00

(52) U.S. Cl. PLT/445

(57) ABSTRACT

A new and distinct Lavandula cultivar named ‘Voodoo’ is disclosed, characterized by a long, dark purple terminal bracts with a broad angle of attachment. The new variety can also be characterized its round plant shape and long peduncle length. The new variety is a Lavandula, normally produced as an outdoor garden or container plant.

### LATIN NAME OF THE GENUS AND SPECIES

Lavandula stoechas

### VARIETY DENOMINATION

‘Voodoo’

### BACKGROUND OF THE INVENTION

The new Lavandula cultivar is a product of a chance discovery by the inventors, Luen Miller and Manuel Morales. The new variety was discovered at a commercial nursery belonging to the inventors, in Watsonville, Calif.

The new variety was selected during Spring of 2011 as a seedling of unknown parentage. The seedling was discovered among plantings of Lavandula stoechas ‘Dedication’, unpatented and Lavandula stoechas ‘Otto Quast’, unpatented. The inventors presume these two varieties are the parent varieties.

Asexual reproduction of the new cultivar was first performed by vegetative cuttings December 9, 2011 at the same commercial nursery in Watsonville, Calif. Subsequent propagation has shown that the unique features of this cultivar are stable and reproduced true to type through successive generations.

### SUMMARY OF THE INVENTION

The cultivar ‘Voodoo’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Voodoo’ These characteristics in combination distinguish ‘Voodoo’ as a new and distinct Lavandula cultivar:

1. Dark purple infertile bract coloration.

2. Long length of infertile bracts.

3. Round plant form.

4. Wide angle of attachment and attitude of infertile bracts.

5. Long peduncle length.

6. Straight peduncles.

7. Strong, durable flowers.

Plants of the new cultivar ‘Voodoo’ are similar to plants of the presumed parent, Lavandula stoechas ‘Dedication’ in most horticultural characteristics, however, plants of the new cultivar ‘Voodoo’ produce flowers of a darker purple. Additionally, the flowers last longer and produce infertile bracts that have a wider angle of attachment and attitude than ‘Dedication’.

Plants of the new cultivar ‘Voodoo’ are similar to plants of the presumed parent; Lavandula stoechas ‘Otto Quast’ in most horticultural characteristics, however, plants of the new cultivar ‘Voodoo’ produce flowers of a different color. Additionally, plants of the new variety produce infertile bracts that have a wider angle of attachment and attitude than ‘Otto Quast’.

### COMMERCIAL COMPARISON

Plants of the new cultivar ‘Voodoo’ are comparable to the variety Lavandula ‘Larkman Bee’, 20840. The two Lavandula varieties are similar in most horticultural characteristics; however, the new variety ‘Voodoo’ differs in producing longer infertile bracts, longer peduncles and an overall longer flower than the comparator. Additionally ‘Voodoo’ produces a plant of stronger vigor and larger mature size than the comparator.

Plants of the new cultivar ‘Voodoo’ can also be compared to the commercial variety Lavandula ‘Larkman Hazel’ U.S. Plant Pat. No. 20,894. These varieties are similar in most horticultural characteristics however, the new variety ‘Voodoo’ differs in producing longer infertile bracts, longer peduncles and an overall longer flower than the comparator. Additionally ‘Voodoo’ produces a plant of stronger vigor and larger mature size than the comparator.

### BRIEF DESCRIPTION OF THE PHOTOGRAPHS

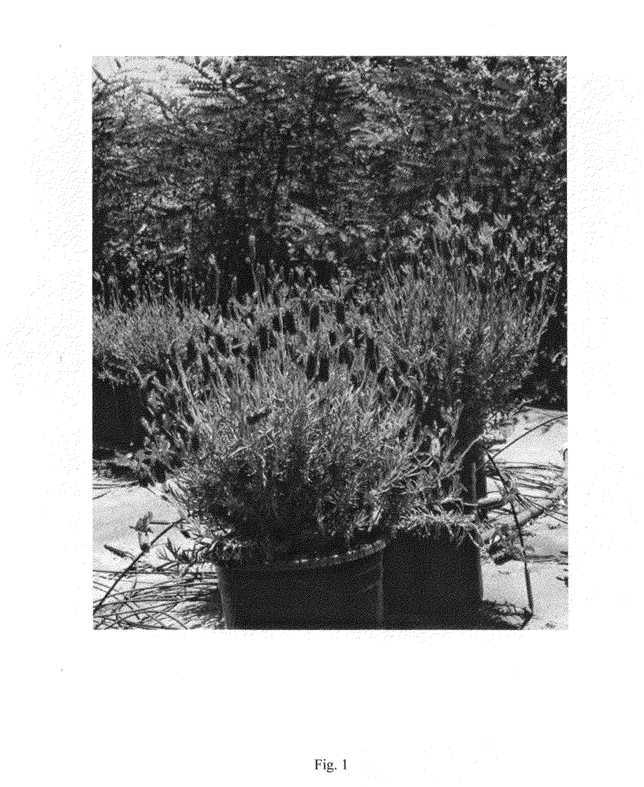

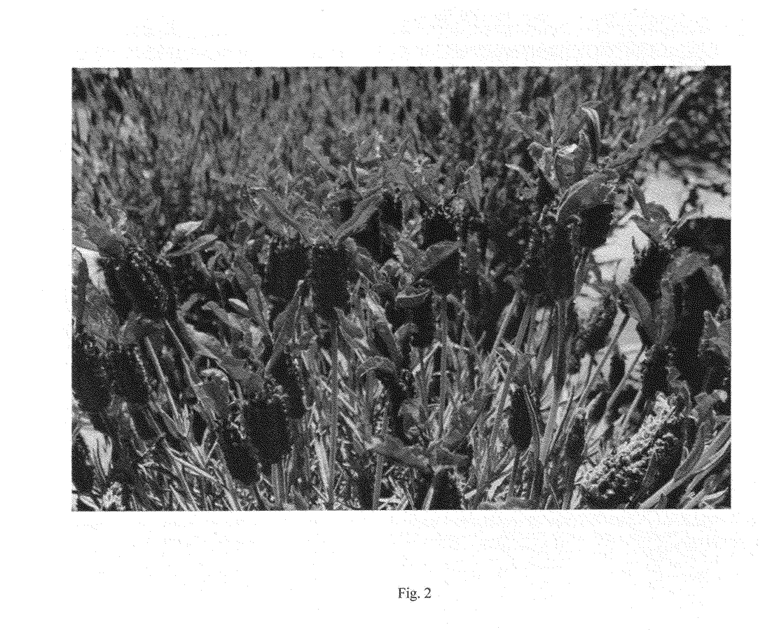

The accompanying photograph in FIG. 1 illustrates in full color a typical plant of ‘Voodoo’ grown outdoors in Watsonville, Calif. The prominent plant in the foreground is ‘Voodoo’. The plant in the background, with a less regular, round shape is Lavendula ‘Dedication’. Age of the plants photographed is approximately 50 weeks from a rooted cutting. FIG. 2 illustrates a close up of the flower. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘Voodoo’ plants grown outdoors during, Fall, Winter and Spring in Watsonville, Calif. The growing temperature ranged from 10° C. to 35° C. during the day and from 5° C. to 25° C. during the night. General light conditions are bright, normal sunlight. Measurements and numerical values represent averages of typical plant types.
Botanical classification: Lavandula stoechas ‘Voodoo’
Propagation:
   Time to initiate roots.—About 10 to 14 days at approximately 21° C.
   Time to produce a rooted cutting.—About 25 to 35 days at 21° C.
Plant:
   Age of plant described.—Approximately 50 weeks from rooted cutting.
   Plant spread.—Approximately 60 cm.
   Plant height.—Approximately 50 cm, to top of flowering plane. Approximately 30 cm to top of foliar plane.
   Growth rate.—Moderate to fast.
   Length of primary lateral branches.—Approximately 16 cm.
   Diameter of lateral branches.—Approximately 0.35 cm.
   Quantity of lateral branches.—About 50.
   Plant habit.—Moderately dense, rounded ball shape.
   Stem.—Juvenile Color: Near Yellow-Green group RHS 145B Mature Color: Near Yellow-Green 147D Texture/Pubescence: Densely pubescent.
   Internode length.—Average 1.0 cm.
Foliage:
   Leaf.—Arrangement: Opposite Leaf type: Simple Quantity: Average 16 fully expanded per main branch and 20 juvenile leaves. Average Length: Average 3.4 cm. (mature foliage) Average Width: Average 0.4 cm. (mature foliage) Shape of blade: Lanceolate Apex: Acute Base: Truncate Attachment: Sessile Margin: Entire Texture of all surfaces: Pubescent Leaf fragrance: Very strong Lavender scent. Color: Young foliage upper side: Near Yellow-Green 147B Young foliage under side: Near Green RHS 137D Mature foliage upper side: Near Green RHS 137B Mature foliage under side: Near Green RHS 137A Venation: Type: Reticulate Venation color upper side: Near Yellow-Green 144A, only main vein distinguishable from foliage color. Venation color under side: Near Yellow-Green 145B Petiole: Absent.
Flower:
   Bloom period.—Main flush late winter to late spring. Lesser quantities of flowers produced throughout Summer and Fall.
   Inflorescence.—Form: Small single flower in verticillasters arranged in spikes. Most commonly 8 rows of flowers. Infrequently 6 or 10 rows. Flowers have small bracts, and large showy terminal bracts. Number of individual flowers per spike: Average 144. Fragrance: Very strong lavender scent Corolla shape: Salverform Petal Number: 5 Petals Fused or Unfused: Fused at base. Petal Shape: Top 2 lobes obovate, lower 3 lobes ovate. Petal Margin: Entire Petal Surfaces: Glabrous Length: Approximately 0.8 cm. (corolla tube) Diameter: Approximately 0.3 cm. Inflorescence Size, excluding terminal bracts: Length: Approximately 5.5 cm. Width: Approximately 1.8 cm. Shape: Cylindrical Coloration of individual flowers, excluding terminal bracts: Immature: Near Violet-Blue N89B Mature: Near Violet-Blue N89B, base N155D Fading: Near Violet-Blue N92B, base N155D Calyx Width: 0.2 cm Calyx Length: 0.95 cm Calyx Shape: Flattened tube. Calyx color: RHS Yellow-Green 144C Bract shape: Elliptic Bract apex: Acute with a cusp. Bract Color: Near Yellow-Green 144C , cusp colored Greyed-Purple 187A.
   Terminal bracts (sterile).—Quantity: 4 large and 1 or 2 smaller per inflorescence.
   Large bracts.—Attitude and angle of attachment: At least 2 of the large bracts are attached at an obtuse angle, near 75°. Terminal bracts and an upward and outwardly angled attitude. Length: Approximately 3.5 to 4.5 cm. Width: Approximately 1.0 cm. Form: Petaloid Margin: Entire and undulating. Surface: Smooth Appearance: Matte. Shape: Oblanceolate Apex: Acute Base: Attenuate. Vein Pattern: Reticulate Duration on Plant: Approximately 12 weeks, with good color. Persistent. Color: Upper side of immature bract: Near RHS Violet 83C, base lighter, 83D. Veins darker, near Violet-Blue N92A Under side of immature bract: Near RHS Violet 83B, base lighter, 83C. Veins darker, near Violet-Blue N92A Upper side of mature bract: Near RHS Violet 83B Under side of mature bract: Near RHS Violet 83A Bract color fading to: Near RHS Purple 79B.
   Small bracts.—Quantity: typically 1 or 2 Length: Approximately 2.0 to 3.0 cm. Width: Approximately 0.8 cm, unfolded. Apex: Shallow bi-lobe Margin: Entire, undulating Base: Attemiate Aspect: Undulating and folded inward at the mid-vein. Duration on Plant: Approximately 12 weeks. Persistent Color: Upper side of immature bract: Near RHS Violet 83C, base lighter, 83D. Veins darker, near Violet-Blue N92A Under side of immature bract: Near RHS Violet 83B, base lighter, 83C. Veins darker, near Violet-Blue N92A Upper side of mature bract: Near RHS Violet 83B Under side of mature bract: Near RHS Violet 83A Bract color fading to: Near RHS Purple 79B.
Peduncle: Peduncle length: Average range approximately 9 to 11 cm. Peduncle diameter: Approximately 0.4 cm. Aspect: Very straight Color: Near RHS Green 138C.
Reproductive organs:
   Androecium.—Stamens: 4. Anther shape: linear. Anther length: Approximately 0.05 cm. Anther color: Near RHS White N155A Pollen quantity: Pollen production not observed.
   Pistil.—Number: 1 Length: Approximately 0.4 cm Style: Length: Approximately 0.3 cm. Color: Near RHS White N155A Stigma: Shape: Linear. Color: Near RHS Violet-Blue N92B Ovary: Minute and immeasurable.
Other characteristics:
   Seeds and fruits.—Seed production not observed to date.
   Disease/pest resistance.—Neither resistance nor susceptibility to the normal diseases and pests of Lavandula stoechas. has been observed.
   Drought tolerance and cold tolerance.—Hardy perennial. Tolerates high temperature to at least 32° C. Tolerates low temperatures to −° 12C. While specific drought tolerance has not been observed, once plants are established very little water is necessary to maintain plants.
   Fruit/seed production.—Fruit and seed production not observed.

What is claimed is:

1. A new and distinct cultivar of Lavandula plant named ‘Voodoo’ as herein illustrated and described.
